(19) United States
(12) Patent Application Publication
(10) Pub. No.: US 2023/0310827 A1
(43) Pub. Date: Oct. 5, 2023

(54) GAS-LIQUID DELIVERY CONNECTOR, GAS-LIQUID DELIVERY CONNECTOR PAIR, AND GAS-LIQUID DELIVERY DEVICE

(71) Applicant: SUZHOU YIBEN LIFE TECHNOLOGY CO., LTD, Suzhou (CN)

(72) Inventor: Yonghua DONG, Solon, OH (US)

(73) Assignee: SUZHOU YIBEN LIFE TECHNOLOGY CO., LTD, Suzhou (CN)

(21) Appl. No.: 18/152,150

(22) Filed: Jan. 10, 2023

Related U.S. Application Data
(63) Continuation of application No. PCT/CN2021/104714, filed on Jul. 6, 2021.

(30) Foreign Application Priority Data
Jul. 10, 2020 (CN) 202021352778.6

(51) Int. Cl. A61M 39/10; F16L 37/58

(52) U.S. Cl. CPC A61M 39/105; F16L 37/58

(57) ABSTRACT

A gas-liquid delivery connector, a gas-liquid delivery connector pair, and a gas-liquid delivery device. The gas-liquid delivery connector comprises: a male connector (1) for connecting a catheter, wherein the male connector (1) is provided with a first connecting channel (11) in the axial direction thereof; and a female connector (2) for connecting a microcatheter, wherein the female connector (2) is provided with a second connecting channel (21) in the axial direction thereof. The female connector (2) is connected to the male connector (1) such that the second connecting channel (21) is in butt joint with and in communication with the first connecting channel (11), and the inner diameter of the second connecting channel (21) is the same as the inner diameter of the first connecting channel (11).

### BACKGROUND

### Technical Field

The present disclosure relates to a gas-liquid delivery connector, a gas-liquid delivery connector pair, and a gas-liquid delivery device, belonging to the technical field of medical instruments.

### Related Art

A Luer connector is an internationally standardized micro-impermeable connector which is formed by connecting a male connector with a matched female connector, and is widely used in medical instruments and laboratory instruments in various countries at present.

In a current Luer connector, the inner diameters of channels of the male Luer connector and the female Luer connector are inconsistent. Generally, the inner diameter of the female Luer connector is large, and the inner diameter of the male Luer connector is small. Through such design, as shown in the circle in FIG. 7, when a liquid is injected into the female Luer connector from the male Luer connector in the direction of the arrow in the figure, the liquid can be prevented from leaking out from the position of butt joint between the two connectors. In Luer connector standards for liquid delivery, one of the detection requirements is that no bubbles occur (except for the first 5 s).

### SUMMARY

A technical problem to be solved by the present disclosure is to provide a gas-liquid delivery connector.

Another technical problem to be solved by the present disclosure is to provide a gas-liquid delivery connector pair. A further technical problem to be solved by the present disclosure is to provide a gas-liquid delivery device.

In order to achieve the above technical objectives, the present disclosure adopts the following technical solutions:

A gas-liquid delivery connector includes:
a male connector for connecting a catheter, wherein the male connector is provided with a first connecting channel in the axial direction thereof; and
a female connector for connecting a microcatheter, wherein the female connector is provided with a second connecting channel in the axial direction thereof.

The female connector is connected to the male connector such that the second connecting channel is in butt joint with and in communication with the first connecting channel, and the inner diameter of the second connecting channel is the same as the inner diameter of the first connecting channel.

Preferably, the end of the male connector far away from the female connector is further provided with a third connecting channel in the axial direction of the male connector, the third connecting channel is in communication with the first connecting channel, and the inner diameter of the third connecting channel is larger than the inner diameter of the first connecting channel.

Preferably, the first connecting channel and the third connecting channel are both located inside the male connector. Preferably, a transition section with a gradually reduced inner diameter is arranged between the first connecting channel and the third connecting channel; and
the first connecting channel, the third connecting channel and the transition section are all located inside the male connector.

Preferably, the second connecting channel has a gradually reduced inner diameter which is gradually reduced from the same inner diameter as that of the first connecting channel to a standard inner diameter of the microcatheter.

A gas-liquid delivery connector pair includes:
a male connector for connecting a catheter, wherein the male connector is provided with a first connecting channel in the axial direction thereof; and
a female connector for connecting a microcatheter, wherein the female connector is provided with a second connecting channel in the axial direction thereof.

The inner diameter of the first connecting channel is the same as the inner diameter of the second connecting channel of the female connector.

Preferably, the male connector further includes a third connecting channel penetrating through the first connecting channel, the third connecting channel is located at the end of the male connector far away from the position in butt joint with the female connector, and the inner diameter of the third connecting channel is larger than the inner diameter of the first connecting channel to perform butt joint with the catheter.

Preferably, the first connecting channel and the third connecting channel are both located inside the male connector and penetrate through the male connector in the axial direction thereof.

Preferably, the length of the third connecting channel is less than the length of the first connecting channel.

A gas-liquid delivery device includes a catheter, a microcatheter and the above-mentioned gas-liquid connector. The catheter is in communication with the first connecting channel of the male connector, and the microcatheter is in communication with the second connecting channel of the female connector.

Compared with the prior art, the present disclosure has the following beneficial effects:

In the gas-liquid connector provided by the present disclosure, since the inner diameter of the first connecting channel of the male connector is the same as the inner diameter of the second connecting channel of the female connector, the sizes of the spaces in the channels are the same. When a gas-liquid mixture flows from the first connecting channel of the male connector to the second connecting channel of the female connector, the gas in the mixture will not gather to avoid the occurrence of large bubbles, so as to ensure the smoothness of flow of the gas-liquid mixture and the uniform distribution of the gas in the liquid, such that a gas-liquid mixed medicine can enter the body smoothly and safely without embolism. In the present disclosure, the position of inner diameter change (from a large inner diameter to a small inner diameter) is set inside the male connector or the female connector instead of the position of butt joint, so that the air tightness is better. Moreover, the inner diameter is gradually reduced, which is conducive to the stable flow of the gas-liquid mixture.

### BRIEF DESCRIPTION OF THE DRAWINGS

FIG. 1 shows a schematic structural view of butt joint of a gas-liquid connector in a first embodiment of the present disclosure.

FIG. 2 shows a schematic cross-sectional structural view of butt joint of the gas-liquid connector.

FIG. 3 shows a schematic cross-sectional structural view of a male connector.

FIG. 4 shows a schematic cross-sectional structural view of a female connector.

FIG. 5 shows a schematic structural view of a gas-liquid delivery device in the first embodiment of the present disclosure.

FIG. 6A shows a schematic view of pipe diameter change of a connector pair in a second embodiment of the present disclosure.

FIG. 6B shows a schematic view of pipe diameter change of a deformation example of the connector pair in the second embodiment of the present disclosure.

FIG. 7 shows a cross-sectional view of butt joint of existing male and female connectors.

### DETAILED DESCRIPTION

The technical solutions of the present disclosure will be further described in detail below with reference to the accompanying drawings and specific embodiments.

An existing Luer connector is suitable for conveying a pure liquid. However, when the existing Luer connector is used for conveying a gas-liquid mixture, the gas in the gas-liquid mixture will float up to the surface of the liquid after entering a larger inner diameter (female connector) from a smaller inner diameter (male connector), and gather together to form larger bubbles, which will destroy the uniform distribution of the gas in the liquid and cause embolism after entering blood vessels.

Different from the Luer connector in the prior art, the gas-liquid connector of the present disclosure is used for conveying a gas-liquid mixture, so it is necessary to avoid gas from gathering during transmission in a pipeline. The gas and liquid in the gas-liquid mixture are uniformly distributed, such as the interval distribution of a section of liquid and a section of gas (referring to the Chinese Patent Application No. 201880021772.X of the applicant entitled “Chain drug structure and preparation device and storage device thereof”), or the gas-liquid mixture is a suspension (ultrasonic contrast agent), etc. composed of a large number of microbubbles dispersed in the liquid.

### Embodiment 1

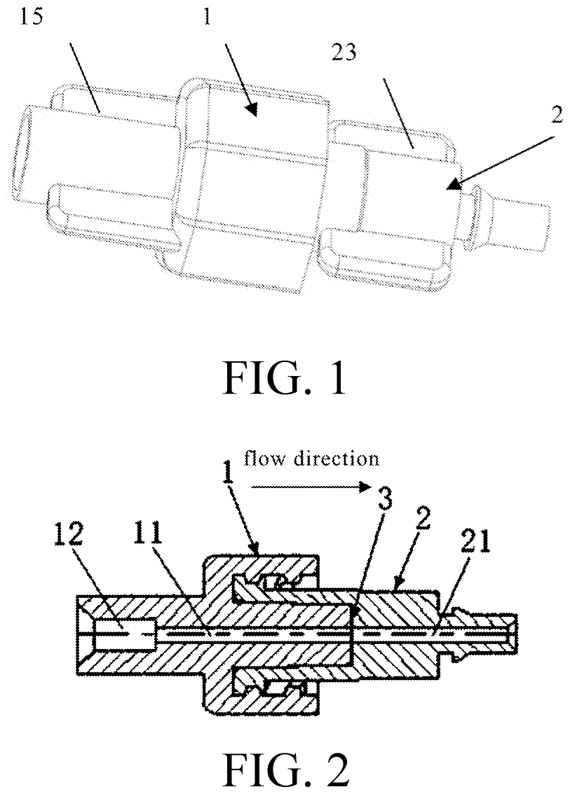

FIG. 1 shows a gas-liquid connector provided in an embodiment of the present disclosure, including: a male connector 1 and a female connector 2. In addition to the structures involved in the following description, the structures of the male connector 1 and the female connector 2 comply with Luer connector standards.

As shown in FIG. 2, the male connector 1 is provided with a first connecting channel 11 at the end in butt joint with the female connector in the axial direction thereof, that is, the downstream position of the flow direction of the liquid (the direction of the arrow in the figure), and is provided with a third connecting channel 12 at the end connected to the catheter in the upstream position of the flow direction.

The female connector 2 is provided with a second connecting channel 21 at the end in butt joint with the male connector 1 in the axial direction thereof, that is, the upstream position of the flow direction of the gas-liquid mixture; and the other end (the downstream position of the flow direction) is used for connecting a microcatheter (the microcatheter can enter the human body). The female connector 2 can be connected to the male connector 1 such that the second connecting channel 21 is in butt joint with and in communication with the first connecting channel 11, and the inner diameter of the second connecting channel 21 is the same as the inner diameter of the first connecting channel 11.

In this embodiment, during specific use, the female connector 2 is connected to the male connector 1, and after the connection is completed, the second connecting channel 21 of the female connector 2 is in butt joint with the first connecting channel 11 of the male connector 1 accordingly, such that the second connecting channel 21 is in communication with the first connecting channel 11. Furthermore, since the inner diameter of the second connecting channel 21 is the same as the inner diameter of the first connecting channel 11 (that is, the sizes of the inner spaces of the first connecting channel 11 and the second connecting channel 21 are the same), when a gas-liquid mixture flows from the first connecting channel 11 of the male connector 1 to the second connecting channel 21 of the female connector 2, the gas in the gas-liquid mixture will not gather to avoid the occurrence of large bubbles, so as to ensure the uniform distribution of the gas and liquid in the gas-liquid mixture and the smoothness of flow, and improve the use effect of the gas-liquid connector.

As shown in FIG. 2, in the above embodiment, preferably, the end of the male connector 1 far away from the position in butt joint with the female connector 2 is further provided with a third connecting channel 12 in the axial direction of the male connector 1 to connect the catheter (for conveying the gas-liquid mixture from the catheter to the gas-liquid connector). The third connecting channel 12 is in communication with the first connecting channel 11, and the inner diameter of the third connecting channel 12 is larger than the inner diameter of the first connecting channel 11. The inner diameter of the third connecting channel 12 is 1.5 mm (slightly larger than the inner diameter of the catheter for enabling the third connecting channel 12 to be in communication with the catheter), and the inner diameter of the first connecting channel 11 is 1.0 mm (slightly larger than the inner diameter of the microcatheter).

Specifically, in this embodiment, since the inner diameter of the third connecting channel 12 is larger than the inner diameter of the first connecting channel 11, when the gas-liquid mixture conveyed to the male connector 1 enters the first connecting channel 11 with a small inner diameter from the third connecting channel 12 with a large inner diameter, the flow rate increases, but no gas escapes. As a result, when the gas-liquid mixture flows to the female connector 2 from the first connecting channel 11 of the male connector 1, since the inner diameter of the first connecting channel 11 is the same as the inner diameter of the second connecting channel 21 of the female connector 2, the flow rate of the gas-liquid mixture will not change, and the gas will not escape from the liquid, so as to realize the smooth flow of the gas-liquid mixture at the joint between the male connector 1 and the female connector 2.

It can be seen that in the male connector 1 in this embodiment, the third connecting channel 12 (the inner diameter of which is larger than the inner diameter of the first connecting channel) and the first connecting channel 11 (the inner diameter of which is the same as the inner diameter of the second connecting channel in the female connector 2) are arranged in sequence in the flow direction, so that the flow state of the gas-liquid mixture changes from the flow state in the channel with a large inner diameter (different from the flow state in the female connector) to the flow state in the channel with a small inner diameter (the same as the flow state in the female connector), and the inner diameter change occurs inside the male connector, which can ensure the air tightness. In contrast, in the prior art, the flow state of the gas-liquid mixture changes from the flow state in the channel with a small inner diameter (the flow state in the male connector) to the flow state in the channel with a large inner diameter (the flow state in the female connector), and the inner diameter change occurs in the position of butt joint between the male connector and the female connector.

Of course, those skilled in the art can understand that a transition section can be arranged between the third connecting channel 12 (the inner diameter of which is larger than the inner diameter of the first connecting channel) and the first connecting channel 11, so that the inner diameter of the third connecting channel 12 is gradually reduced from the same inner diameter as that of the catheter to the same inner diameter as that of the microcatheter (that is, the inner diameter of the first connecting channel 11). This is more conducive to the smooth flow of the gas-liquid mixture. The first connecting channel 11, the third connecting channel 12 and the transition section are all arranged inside the male connector and substantially penetrate through the entire axial length of the male connector (chamfer parts shown in the figure can be included).

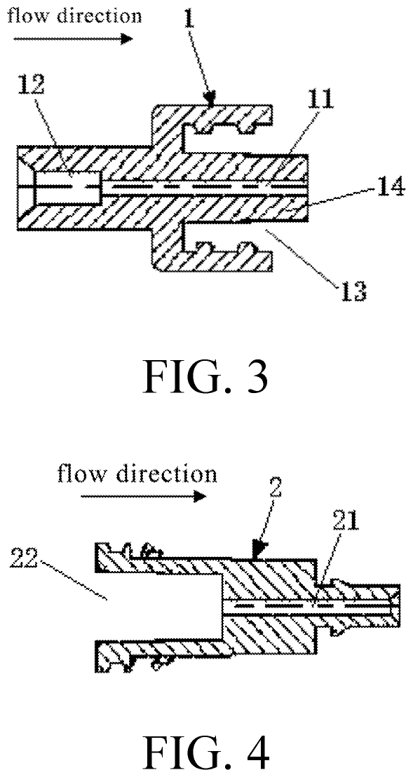

As shown in FIG. 3 and FIG. 4, in the above embodiment, preferably, an end face of the male connector 1 close to the female connector 2 is inward concave to form a locking cavity 13, a convex pillar 14 extends in the locking cavity 13 in the axial direction of the male connector 1, and the first connecting channel 11 penetrates through the convex pillar 14 in the axial direction of the male connector 1. An end face of the female connector 2 close to the male connector 1 is inward concave to form a concave part 22, and the concave part 22 is in communication with the second connecting channel 21. The female connector 2 can be connected to the locking cavity 13 of the male connector 1, so that the convex pillar 14 can be inserted in the concave part 22, and the first connecting channel 11 is in butt joint with and in communication with the second connecting channel 21. As a result, after the male connector 1 and the female connector 2 are connected, the stability of connection between the male connector 1 and the female connector 2 can be improved by means of the mutual cooperation between the convex pillar 14 and the concave part 22.

In the above embodiment, preferably, an inner wall of the locking cavity 13 is provided with internal threads, and an outer wall of the female connector 2 is provided with external threads, so that the female connector 2 can be in threaded connection with the locking cavity 13. In this embodiment, the male connector 1 and the female connector 2 are connected by means of threads, which can facilitate the disassembly and assembly of the gas-liquid connector. However, it should be noted that the threaded connection method in this embodiment is only one of the preferable implementation methods. In other embodiments, the female connector 2 can also be connected to the locking cavity 13 of the male connector 1 by means of buckles, etc.

In the above embodiment, preferably, an outer wall of the male connector 1 is provided with two first lug plates 15 which are arranged oppositely, and the outer wall of the female connector 2 is provided with two second lug plates 23 which are arranged oppositely. When the male connector 1 and the female connector 2 need to be in threaded connection or disassembled, the first lug plates 15 and the second lug plates 23 are respectively rotated clockwise or counterclockwise with both hands, so as to conveniently connect or disassemble the male connector 1 and the female connector 2.

In the above embodiment, preferably, a sealing ring 3 is arranged between the end of the convex pillar 14 and the bottom of the concave part 22. Specifically, the sealing ring 3 can be made of a silicone material, so that the sealing ring 3 has certain elasticity. After the convex pillar 14 is inserted into the concave part 22, the convex pillar 14 will tightly press the sealing ring 3 against the bottom of the concave part 22, so as to seal the gap between the convex pillar 14 and the concave part 22 to prevent the gas-liquid mixture from flowing out from the gap between the convex pillar 14 and the concave part 22, thereby improving the air tightness of the gas-liquid connector.

In the above embodiment, preferably, the convex pillar 14 is in interference fit with the concave part 22. Therefore, the air tightness and the stability of connection between the convex pillar and the concave part 22 are further improved.

In the above embodiment, preferably, the male connector 1 is an integrally formed structure, and the female connector 2 is an integrally formed structure. Specifically, the production of the male connector 1 and the female connector 2 can be completed by means of injection molding, thus avoiding the secondary assembly process and improving the production efficiency.

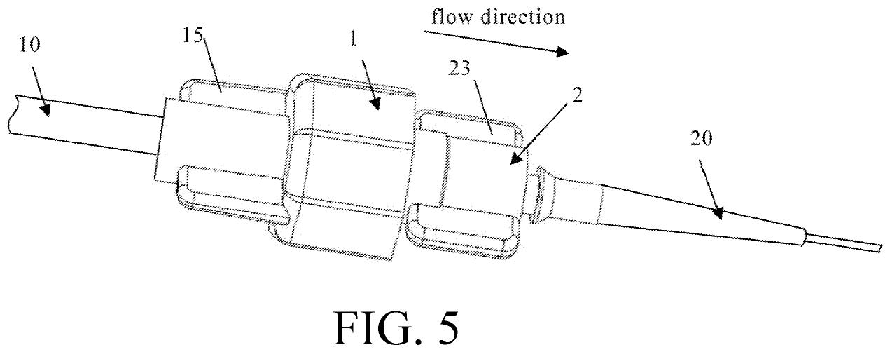

As shown in FIG. 5, an embodiment of the present disclosure further provides a gas-liquid delivery device using the above-mentioned male and female connectors, including a catheter 10, a microcatheter 20 and the gas-liquid connector in Embodiment 1.

The catheter 10 is in communication with the first connecting channel 11 of the male connector 1 to convey the gas-liquid mixture to the male connector 1. The microcatheter 20 is in communication with the second connecting channel 21 of the female connector 2 to convey the gas-liquid mixture from the female connector to the human body.

### Embodiment 2

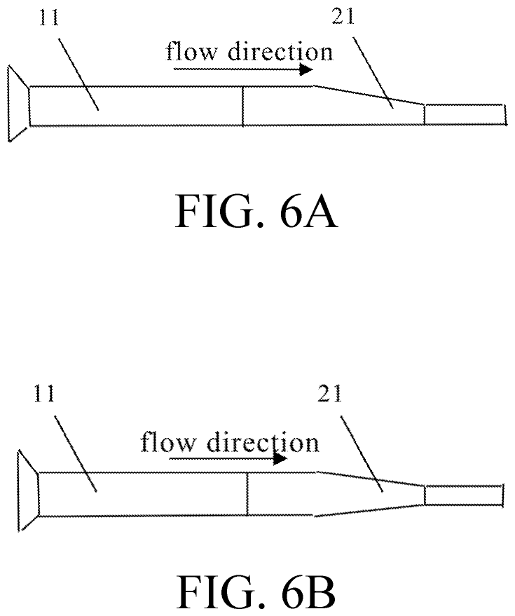

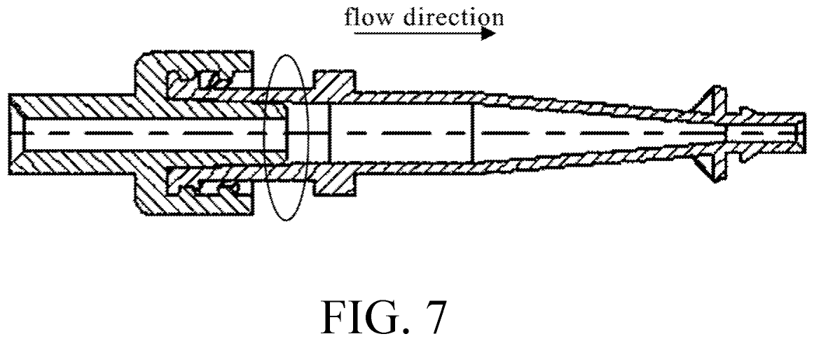

In addition to the structures involved in the following description, the structures of the gas-liquid connector are the same as those in the first embodiment. FIG. 6A and FIG. 6B show schematic views of inner diameter change of a first connecting channel and a second connecting channel of a connector pair structure in a second embodiment. In this embodiment, the male connector can only include the first connecting channel 11, but does not include the third connecting channel 12; and the second connecting channel 21 of the female connector has a variable inner diameter.

Specifically, at one end of the second connecting channel 21, the inner diameter of the position of butt joint between the female connector and the male connector is the same as the inner diameter of the first connecting channel 11; and at the other end of the second connecting channel 21, the inner diameter of the second connecting channel 21 is gradually reduced to the inner diameter of the microcatheter for conveying blood, etc. to the body (such as the standard inner diameter specified in the Chinese national standard GB/T1962.2-2001). As shown in the figure, in this embodiment, the inner diameter of the channel is reduced at the end with the female connector.

In this embodiment, during specific use, the gas-liquid mixture is conveyed to the male connector 1 through the catheter 10, and then, the gas-liquid mixture enters the second connecting channel 21 of the female connector 2 through the first connecting channel 11 of the male connector, and is conveyed to the human body through the microcatheter 20 from the second connecting channel 21 of the female connector 2.

It can be seen that the position of inner diameter change (from a large inner diameter to a small inner diameter) is set inside the male connector or the female connector instead of the position of butt joint, so that the air tightness is better. Moreover, the inner diameter is gradually reduced, which is conducive to the stable flow of the gas-liquid mixture. By means of the gas-liquid connector, the gas in the gas-liquid mixture will not gather to avoid the occurrence of large bubbles, so as to ensure the smoothness of flow of the gas-liquid mixture and the uniform distribution of the gas in the liquid, such that a gas-liquid mixed medicine can enter the body smoothly and safely without embolism.

The technical solutions of the present disclosure are described in detail above. For those skilled in the art, any obvious changes made to the present disclosure without departing from the essence of the present disclosure will constitute an infringement of the patent right of the present disclosure and will bear corresponding legal liabilities.

1. A gas-liquid delivery connector, comprising:
a male connector for connecting a catheter, wherein the male connector is provided with a first connecting channel in the axial direction thereof; and
a female connector for connecting a microcatheter, wherein the female connector is provided with a second connecting channel in the axial direction thereof.
The female connector is connected to the male connector such that the second connecting channel is in butt joint with and in communication with the first connecting channel, and the inner diameter of the second connecting channel is the same as the inner diameter of the first connecting channel.

2. The gas-liquid delivery connector according to claim 1, wherein
the end of the male connector far away from the female connector is further provided with a third connecting channel in the axial direction of the male connector, the third connecting channel is in communication with the first connecting channel, and the inner diameter of the third connecting channel is larger than the inner diameter of the first connecting channel.

3. The gas-liquid delivery connector according to claim 2, wherein
the first connecting channel and the third connecting channel are both located inside the male connector.

4. The gas-liquid delivery connector according to claim 3, wherein
a transition section with a gradually reduced inner diameter is arranged between the first connecting channel and the third connecting channel; and
the first connecting channel, the third connecting channel and the transition section are all located inside the male connector.

5. The gas-liquid delivery connector according to claim 1, wherein
the second connecting channel has a gradually reduced inner diameter which is gradually reduced from the same inner diameter as that of the first connecting channel to a standard inner diameter of the microcatheter.

6. A gas-liquid delivery connector pair, comprising:
a male connector for connecting a catheter, wherein the male connector is provided with a first connecting channel in the axial direction thereof and
a female connector for connecting a microcatheter, wherein the female connector is provided with a second connecting channel in the axial direction thereof.
The inner diameter of the first connecting channel is the same as the inner diameter of the second connecting channel of the female connector.

7. The gas-liquid delivery connector pair according to claim 6, wherein
the male connector further comprises a third connecting channel penetrating through the first connecting channel, the third connecting channel is located at the end of the male connector far away from the position in butt joint with the female connector, and the inner diameter of the third connecting channel is larger than the inner diameter of the first connecting channel to perform butt joint with the catheter.

8. The gas-liquid delivery connector pair according to claim 7, wherein
the first connecting channel and the third connecting channel are both located inside the male connector and penetrate through the male connector in the axial direction thereof.

9. The gas-liquid delivery connector pair according to claim 7, wherein
the length of the third connecting channel is less than the length of the first connecting channel.

10. A gas-liquid delivery device, comprising a catheter, a microcatheter and the gas-liquid connector according to claim 1, wherein the catheter is in communication with the first connecting channel of the male connector, and the microcatheter is in communication with the second connecting channel of the female connector.
